(19) United States
(12) Patent Application Publication
(10) Pub. No.: US 2021/0346898 A1
(43) Pub. Date: Nov. 11, 2021

(54) Suspended Thermal Radiator Vaporizing Assembly

(71) Applicant: GS Holistic, LLC, Los Angeles, CA (US)

(72) Inventor: Michael Trzecieski, Parry Sound (CA)

(21) Appl. No.: 17/315,286

(22) Filed: May 8, 2021

Related U.S. Application Data
(60) Provisional application No. 63/022,353, filed on May 8, 2020.

(51) Int. Cl. B05B 9/00; B05B 9/01; B05B 15/65

(52) U.S. Cl. CPC B05B 9/002; A61M 2021/0016; B05B 15/65; B05B 9/01

(57) ABSTRACT

A novel vaporizing assembly is disclosed using a suspended thermal radiator that is spaced apart from a phyto material support surface facing a proximal end of the housing and accessible through the proximal opening and fluidly coupled therewith, the phyto material support surface configured to support a phyto material extract for vaporizing and disposed distally below the proximal opening and in fluid communication therewith. The phyto material support surface for receiving of the phyto material extract for vaporizing for having at least a portion of the phyto material extract gravitationally induced to flow towards a pooling area, the phyto material extract proximate the pooling area for thermally contacting the heating element assembly and for being heated to a predetermined temperature to result in a vapor to be emitted from the phyto material extract. A lid assembly is provided for directing airflow from an outside environment towards the phyto material in the pooling area.

### CROSS REFERENCE TO RELATED APPLICATION

This application claims the benefit of U.S. Provisional Application 63/022,353 filed on May 8, 2020, the disclosure of which are incorporated herein.

### FIELD OF THE INVENTION

The technical field relates to a device for vaporization of phyto materials and more specifically to a device for vaporization of phyto material extracts.

### INTRODUCTION

The following is intended to introduce the reader to the detailed description that follows and not to define or limit the claimed subject matter. Aromatherapy generally uses essential oils, which are extracted from phyto materials, such as leaves of plants, for therapeutic benefits. These essential oils are either massaged into the skin or can be inhaled. In some cases the phyto materials are heated in order to released the essential oils therefrom. By heating these phyto materials at predetermined temperatures, essential oils and extracts are boiled off, depending upon the temperature at which these phyto materials are heated, an aroma or vapor is given off, which is then inhaled by a user for its therapeutic benefits. Devices that provide such operation are generally known as vaporizers. There are also extracts available that are derived from the phyto material or loose-leaf aromatherapy materials and these have a consistency of honey and are typically highly purified forms. Normally these extracts are vaporized at temperatures between 500 to 700 degrees Fahrenheit.

Devices that process these concentrated phyto material extracts typically include a waterpipe, or water filtration apparatus, that has an input port and an inhalation aperture with a fluid pathway formed therebetween. Phyto material extract is applied to the vaporization element and a user inhales from the inhalation aperture of the waterpipe, which results in vaporized phyto material and ambient air to flow into the inhalation aperture and into the fluid pathway for being cooled by the water which is typically disposed within this fluid pathway to cool the vapor air mixture.

Conventional vaporizing devices that are used for vaporizing extract materials mostly utilize a bottom heating system which heats a phyto material extract contact surface from the bottom. The disadvantage of the bottom heating system heating that material that is placed onto the extract contact surface may burn due to prolonged contact with the bottom of the heating system. It would be advantageous to provide for a system and method of vaporizing of phyto material extracts that does not heat the phyto material extracts from the bottom that causes materials to be burned or overheated.

It is therefore an object of the invention to provide an aromatherapy vaporization device that overcomes the aforementioned deficiencies.

### SUMMARY

The following introduction is provided to introduce the reader to the more detailed description to follow and not to limit or define any claimed or as yet unclaimed invention. One or more inventions may reside in any combination or sub-combination of the elements or process steps disclosed in any part of this document including its claims and figures.

In accordance with an aspect of this disclosure, there is provide a suspended thermal radiator vaporizing assembly comprising: a housing having a proximal end and a distal end, the distal end disposed lower than the proximal end and the proximal end comprising a proximal opening and at the distal end comprising a distal opening; a phyto material support surface facing the proximal end of the housing and accessible through the proximal opening and fluidly coupled therewith, the phyto material support surface configured to support a phyto material extract and disposed distally below the proximal opening and in fluid communication therewith, a heating element assembly disposed between the proximal opening and the phyto material support surface the heating element assembly comprising a proximal end and a distal end, the distal end of the heating element assembly being proximally spaced from the phyto material support surface through a heating element assembly gap, the distal end of the heating element assembly for providing of at least one of convective and conductive heating to the phyto material extract; the phyto material support surface for receiving of the phyto material extract for vaporizing for having at least a portion of the phyto material extract gravitationally induced to flow towards a pooling area, the phyto material extract proximate the pooling area for thermally contacting the heating element assembly and for being heated to a predetermined temperature to result in a vapor to be emitted from the phyto material extract; a fluid pathway formed within the housing and fluidly coupled with the pooling area through at least a vapor aperture and towards the distal opening at the distal end of the housing, wherein, in use, the vapor flows along the fluid pathway towards the distal end of the housing.

In accordance with an aspect of this disclosure, there is provided a suspended thermal radiator vaporizing assembly comprising: disposing and supporting of the vaporizer assembly into a downstem of a water pipe so that a portion of the housing of the vaporizer assembly heating supported by a water pipe downstem; inserting phyto material extract through a proximal opening with the phyto material extract touching the phyto material support surface and at least a portion of the inserted phyto material extract for gravitationally flowing along the phyto material support surface towards a pooling area; providing a heating element assembly spaced proximally from the phyto material support surface through a heating element assembly gap, applying of electrical energy to the heating element assembly, the heating element assembly for providing of thermal energy to material for vaporizing proximate the heating element assembly through at least one of convective and conductive heating so that at least a portion of the material for vaporizing heating vaporized into a vapor; and drawing the vapor along a vapor path proximate the phyto material support surface and a distal end of the heating element assembly and through the housing and into the downstem of the water pipe, wherein in use the phyto material support surface other than achieves a temperature sufficient to result in vaporization of the phyto material extract.

In accordance with an aspect of this disclosure, there is provided a suspended thermal radiator vaporizing assembly comprising: a housing having a proximal end and a distal end, the distal end disposed lower than the proximal end and the proximal end comprising a proximal opening and at the distal end comprising a distal opening; a phyto material support surface facing the proximal end of the housing and accessible through the proximal opening and fluidly coupled therewith, the phyto material support surface configured to support a phyto material extract for vaporizing and disposed distally below the proximal opening and in fluid communication therewith, a heating element assembly disposed between the proximal opening and the phyto material support surface the heating element assembly comprising a proximal end and a distal end, the distal end of the heating element assembly being proximally spaced from the phyto material support surface through a heating element assembly gap, the distal end of the heating element assembly for providing of at least one of convective and conductive heating to the phyto material extract; the phyto material support surface for receiving of the phyto material extract for vaporizing for having at least a portion of the phyto material extract gravitationally induced to flow towards a pooling area, the phyto material extract proximate the pooling area for thermally contacting the heating element assembly and for being heated to a predetermined temperature to result in a vapor to be emitted from the phyto material extract; a lid assembly, the lid assembly for being releasably coupled with the housing for being in an uncoupled state with the housing for loading of phyto material extract into a phyto material receiving cavity; the lid assembly for being releasably coupled with the housing for being in a coupled state with the housing for having the lid assembly for rotating about a heating element assembly longitudinal axis; and the lid assembly comprising an airflow directing member for receiving of external air from an outside environment into at least a vapor aperture and a vapor output aperture disposed within the phyto material receiving cavity and proximate the phyto material support surface whereby air deflects from the vapor output aperture upon impacting of the phyto material support surface and a controllable airflows stream may be directed towards the phyto material in the pooling area; and a fluid pathway formed within the housing and fluidly coupled with the pooling area through the at least a vapor aperture and towards the distal opening at the distal end of the housing, wherein, in use, the vapor flows along the fluid pathway towards the distal end of the housing.

In some embodiment a suspended thermal radiator vaporizing assembly is provided having a lid assembly rotationally coupled in relation with the phyto material support surface for rotating about a heating element assembly longitudinal axis; the lid assembly comprising an airflow directing member for receiving of external air from an outside environment into at least a vapor aperture and a vapor output aperture disposed within a phyto material receiving cavity and proximate the phyto material support surface whereby air deflects from the vapor output aperture upon impacting of the phyto material support surface and a controllable airflows stream may be directed towards the phyto material in the pooling area.

In some embodiment a vaporizing assembly is provided wherein the phyto material support surface comprises at least a partial paraboloid surface that comprises a quadric surface that has one axis of symmetry wherein a proximal end of the phyto material support surface has an outer diameter greater than an outer diameter of a distal end of the phyto material support surface and the phyto material support surface tapers from the proximal to the distal end comprising the pooling area.

In some embodiment a vaporizing assembly is provided wherein the heating element assembly is coupled to the housing with a coupling arm that supports the heating element assembly for providing the heating element assembly gap with the heating element distal end proximate the pooling area.

In some embodiment a vaporizing assembly is provided wherein the heating element assembly gap may be about 0.5 mm.

In some embodiment a vaporizing assembly is provided comprising a lid assembly, the lid assembly for being releasably coupled with the housing, wherein the heating element heating disposed between the lid assembly and the phyto material support surface when the lid is coupled with the housing the proximal opening may be covered for other than inserting of phyto material extract into the vaporizing assembly.

In some embodiment a vaporizing assembly is provided comprising a lid assembly, the lid assembly for being hinged the housing, wherein the lid assembly for operating between a covered position where the lid assembly provides for reduced external airflow from an outside environment into the at least a vapor aperture and an uncovered position where the external airflow may flow from an outside environment into the at least a vapor aperture.

In some embodiment a vaporizing assembly is provided comprising: a lid assembly, the lid assembly for being releasably coupled with the housing for being in an uncoupled state with the housing for loading of phyto material extract into a phyto material receiving cavity; the lid assembly for being releasably coupled with the housing for being in a coupled state with the housing for having the lid assembly for rotating about a heating element assembly longitudinal axis; and the lid assembly comprising an airflow directing member for receiving of external air from an outside environment into at least a vapor aperture and a vapor output aperture disposed within the phyto material receiving cavity and proximate the phyto material support surface whereby air deflects from the vapor output aperture upon impacting of the phyto material support surface and a controllable airflows stream may be directed towards the phyto material in the pooling area.

In some embodiment a vaporizing assembly is provided wherein the heating element assembly comprises a vertical sidewall extending from the proximal end towards the distal end, the vertical sidewall comprising a non porous surface for other than for supporting the phyto material extract and comprising a resistive heating wire embedded therein.

In some embodiment a vaporizing assembly is provided wherein the heating element assembly comprises a vertical sidewall extending from the proximal end towards the distal end, the vertical sidewall comprising a porous surface for and comprising a resistive heating wire embedded therein.

In some embodiment a vaporizing assembly is provided comprising a support unit for releasably coupling with the distal end of the housing, wherein the support unit is for providing of electrical power to the heating element assembly, the support unit for engaging a water pipe downstem so that the vaporizing assembly heating supported on by the water pipe downstem and the distal opening communicates with a lumen of the water pipe downstem.

In some embodiment a vaporizing assembly is provided wherein the heating element assembly comprises a vertical sidewall extending from the proximal end towards the distal end, the vertical sidewall other than supporting the material for vaporizing thereon, wherein the vertical sidewall is manufacture from a non porous material, wherein a resistive heating wire is contained within vertical sidewall that surrounds the resistive heating wire.

In some embodiment a vaporizing assembly is provided comprising a lid assembly, the lid assembly for being releasably coupled with the housing, wherein when the lid assembly is other than coupled with the housing, the coupling arm remains coupled with the housing and the heating element assembly remains in a fixed position in relation to the phyto material support surface.

In some embodiment a vaporizing assembly is provided wherein the heating element assembly is coupled to a first end of the coupling arm and a second end of the coupling arm heating releasably coupled with the housing through an electrical connection port.

In some embodiment a vaporizing assembly is provided wherein the a connection port comprises at least two releasable electrical contacts for coupling of the heating element assembly to an external control circuit assembly.

### BRIEF DESCRIPTION OF THE DRAWINGS

FIG. 1A illustrates a vaporizing assembly from a cutaway perspective view and in accordance with a first embodiment of the invention;

FIG. 1B illustrates a vaporizing assembly from a cutaway side view and in accordance with a first embodiment of the invention;

FIG. 1C illustrates a heating element assembly and a coupling arm from a cutaway perspective view;

FIG. 1D illustrates a lid assembly releasably coupled with a housing and in a closed state;

FIG. 1E illustrates a support unit that may include a housing and may comprise a user interface and may be connected with a vaporizing assembly;

FIG. 2A illustrates a vaporizing assembly from a cutaway perspective view and in accordance with a second embodiment of the invention;

FIG. 2B illustrates a vaporizing assembly from a top perspective view and with a lid opened; and

FIG. 2C illustrates a vaporizing assembly from a side cutaway view and with a lid opened.

### DETAILED DESCRIPTION

Various apparatuses, methods and compositions are described below to provide an example of an embodiment of each claimed invention. No embodiment described below limits any claimed invention and any claimed invention may cover apparatuses and methods that differ from those described below. The claimed inventions are not limited to apparatuses, methods and compositions having all of the features of any one apparatus, method or composition described below or to features common to multiple or all of the apparatuses, methods or compositions described below. It is possible that an apparatus, method or composition described below is not an embodiment of any claimed invention. Any invention disclosed in an apparatus, method or composition described below that is not claimed in this document may be the subject matter of another protective instrument, for example, a continuing patent application, and the applicant(s), inventor(s) and/or owner(s) do not intend to abandon, disclaim, or dedicate to the public any such invention by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures, and components have not been described in detail so as not to obscure the example embodiments described herein. Also, the description is not to be considered as limiting the scope of the example embodiments described herein.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising,” and variations thereof mean “including but not limited to,” unless expressly specified otherwise. A listing of items does not imply that any or all of the items are mutually exclusive, unless expressly specified otherwise. The terms “a,” “an,” and “the” mean “one or more,” unless expressly specified otherwise.

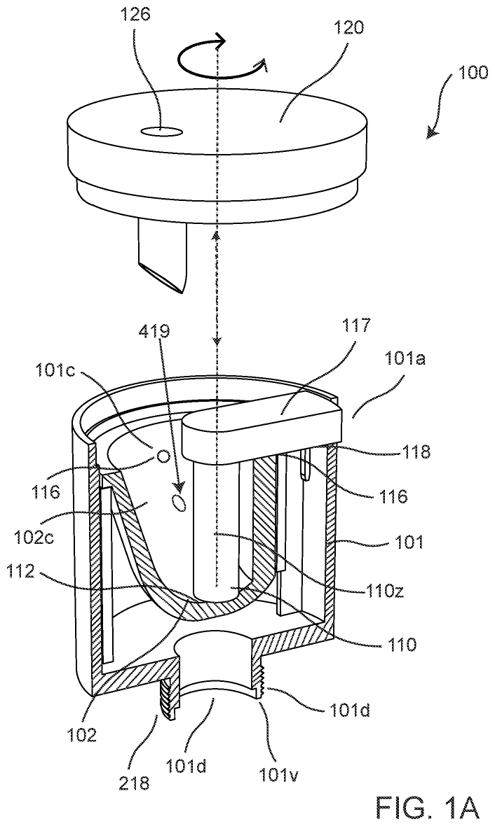

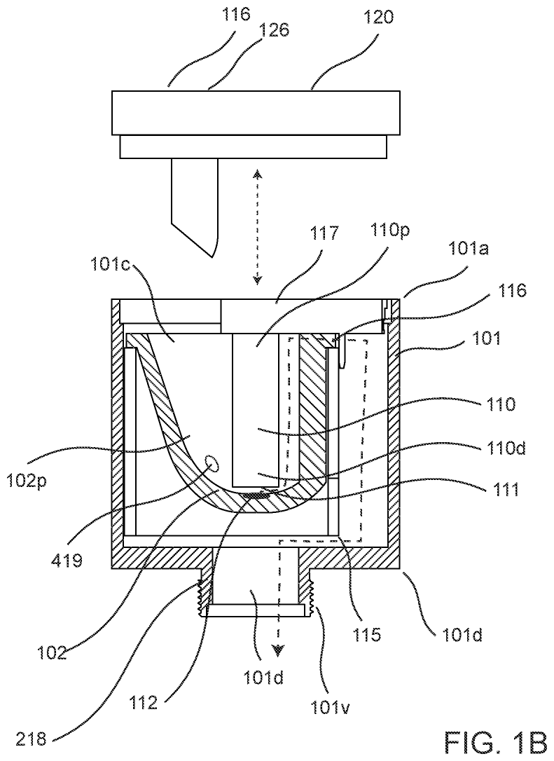

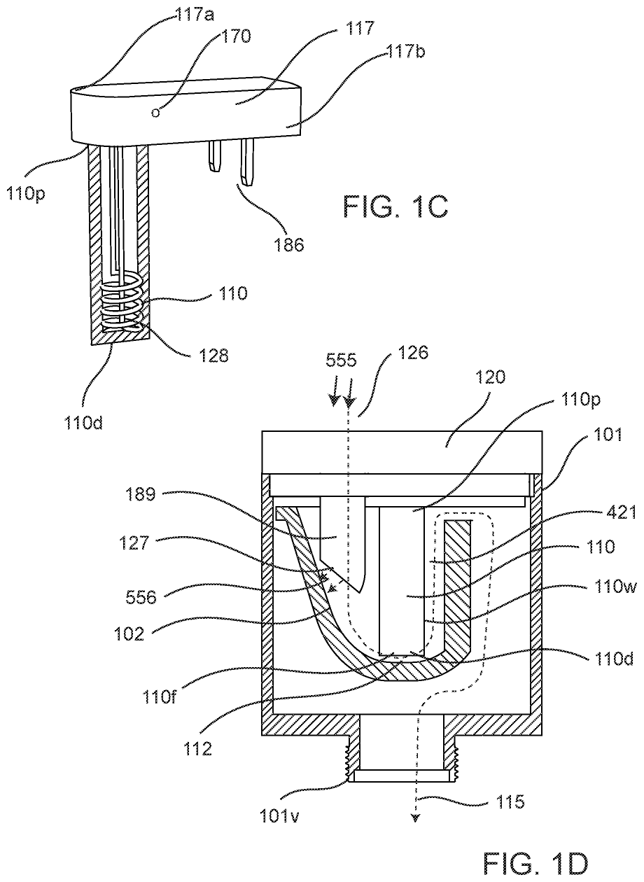

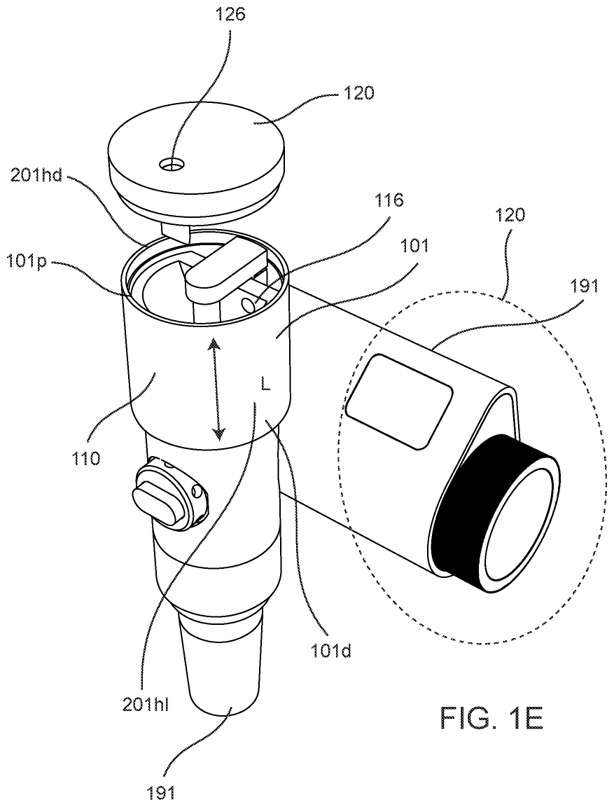

FIGS. 1A through 1E illustrate various aspects of a vaporizing assembly 100 in accordance with a first embodiment of the invention. More specifically, FIG. 1A illustrates, the vaporizing assembly 100 from a cutaway perspective view and in accordance with a first embodiment of the invention. The vaporizing assembly 100 that may be formed from a housing 101 having a proximal end 101a and a distal end 101b, the distal end 101b disposed lower than the proximal end 101a and the proximal end 101a comprising a proximal opening 101c and at the distal end comprising a distal opening 101d. The housing 101 is shown from a cutaway view in FIG. 1A.

FIG. 1B illustrates a lid assembly 120 provided with the lid assembly for being releasably coupled with the housing 101 and the lid assembly 120 being in an open state for loading of phyto material extract through the proximal opening 101c. Referring to FIGS. 1A and 1B, a phyto material support surface 102 may be provided facing the proximal end 101a of the housing 101 and accessible through the proximal opening 101c and fluidly coupled therewith, the phyto material support surface 102 configured to support a phyto material for vaporizing 419 and disposed distally below the proximal opening 101c and in fluid communication therewith. In some embodiments the phyto material support surface 102 may be cup shaped or annular shaped or in some embodiments it may be upwardly curved towards the proximal opening and facing the proximal end 101a of the housing 101. The phyto material support surface 102 proximally facing may form a phyto material receiving cavity 102c.

The phyto material for vaporizing 419 may comprise a phyto material extract. The phyto material support surface 102 is shown from a cutaway view in this figure for clarity. The phyto material extract may have its viscosity reduced with an application of heat thereto.

The phyto material support surface 102 may be for receiving of a phyto material for vaporizing 419 which may be loaded by user through the proximal opening 101c. Upon loading of the phyto material for vaporizing 419, the material may be gravitationally induced to flow towards a pooling area 112. Where the phyto material for vaporizing 419 proximate the pooling area 112 may be contacting the heating element assembly 110 and be heated to a predetermined temperature to result in a vapor 421 to be emitted from the material for vaporizing 419. The pooling area 112 may be approximately centrally disposed with respect to the phyto material support surface 102.

The phyto material support surface 102 may be formed from at least a partial paraboloid surface 102p that comprises a quadric surface that may have one axis of symmetry wherein a proximal end of the phyto material support surface 102 has an outer diameter greater than an outer diameter of a distal end of the phyto material support surface 102 and the phyto material support surface 102 tapers from the proximal to the distal end comprising the pooling area 112. In some embodiments there may also be a semi spherical dish or bowl and there may result in a section of the partial paraboloid surface 102p to be cut by a flat surface. The phyto material support surface 102 may be manufactured from a non-porous material such as a ceramic or a glass or a metal material.

FIG. 1C illustrates a heating element assembly 110 from a cutaway perspective view. Referring to FIGS. 1C and 1D, the heating element assembly 110 may be disposed between the proximal opening 101c and the phyto material support surface 102 the heating element assembly 110 comprising a proximal end 110a and a distal end 110b, the distal end 110b of the heating element assembly 110 being proximally spaced from the phyto material support surface 102 through a heating element assembly gap 111. In some embodiments the gap 111 may be adjustable from 0.2 mm to approximately 3 millimetres or in some cases 1.2 mm.

Referring to FIGS. 1C and 1B, The heating element assembly 110 may be coupled to the housing 101 with a coupling arm 117 that extends radially towards a center of the paraboloid surface as shown in FIG. 1B. The coupling arm 117 may support the heating element assembly 110 for facilitating a creation of the heating element assembly gap 111. The gap 111 may be formed between the distal end of the heating element assembly 110 and the proximally facing phyto material support surface 102.

The coupling arm 117 may be formed from a thermal insulating material and coupling arm may include at least a vapor channel that is for incoming air and in some embodiments for outgoing vapor. The coupling arm may include coupling arm contacts 186 for electrically coupling of the heating element with a control circuit assembly (not shown).

Referring to FIG. 1D, the lid assembly 120 may be provided with the lid assembly being releasably coupled with the housing 101 and the lid assembly 120 being in a closed state for other than loading of phyto material extract through the proximal opening 101c. A fluid pathway 115 may be formed within the housing 101 from the pooling area 112 through at least a vapor aperture 116 and towards the distal opening 101d at the distal end of the housing 101, wherein the vapor 421 may flow along the fluid pathway 115.

The heating element may be disposed between the lid assembly 120 and the phyto material support surface 102 when the lid 120 may be coupled with the housing 101 and the proximal opening 101c may be covered. The lid assembly may be magnetically or frictionally or coupled through a hinge mechanism with the housing 101. The lid assembly 120, may alter airflow characteristics about the distal end of the heating element assembly when coupled with the housing 101.

In some embodiments the lid assembly may be hinged with the housing 101. The lid assembly 120 may be for operating between a covered position where the lid assembly 120 may be for reduced external airflow from an outside environment 555 and for fluidly interacting with the vapors for the combined external airflow and vapors to flow into the at least a vapor aperture 126 where the external air 555 may flow from an outside environment into the at least a vapor aperture 126 and internal flow directed against the phyto material support surface 102 from a vapor output aperture 127 whereby air deflects upon impacting of the phyto material support surface 102. In an uncovered position or loading of phyto material extract into a phyto material receiving cavity.

Referring to FIG. 1D, the heating element assembly 110 may include a vertical sidewall 110w extending from the proximal end 110p towards the distal end 110d, the vertical sidewall 110w other than for supporting the phyto material for vaporizing 419 and for contacting of the phyto material for vaporizing 419 disposed proximate the pooling area 112. The vertical sidewall 110w may be non-porous proximate the proximal end of the heating element assembly to facilitate a flow of material distally towards the distal end and towards the pooling area. The vertical sidewall 110w may have a cylindrical cross section and may include a floor proximate and facing the pooling area.

In some embodiments, material for vaporizing 419 may be disposed towards the proximal end of the material support surface 102 and it may gravitate towards the distal end of material support surface 102 after the phyto material for vaporizing 419 is inserted into the housing 101 from the proximal end and contacts phyto material support surface 102 and may slide along the vertical sidewall 110w under an influence of gravity. The material for vaporizing 419, when gravitating along the material support surface 102, and in some embodiments may gravitate along the along the vertical sidewall 110w and may pool at a centre of the material support surface 102 proximal or at least partially disposed within the pooling area 112.

In some embodiments, the distal end of the heating element assembly may physically contact the pooled material and in some embodiments the pooled material may be thermally coupled with the distal end of the heating element assembly 110. Thermal characteristics of the distal end of the heating element assembly may be affected by a depth and volume of phyto material extract loaded into the housing 101 proximate the pooling area.

Referring to FIG. 1C, the distal end of the heating element assembly 110, may include a resistive heating wire 128, that may have a resistance of approximately 0.3 to 0.5 ohms. The resistive heating wire 128, may heat with a power of approximately 15 watts to 45 Watts. The heating may result in the heating element assembly distal end, or tip, to heat to a temperature of approximately 600 degrees Fahrenheit in approximately 30 to 45 seconds. The distal end, of the heating element assembly 110b, may radiate heat towards the phyto material for vaporizing 419 disposed proximate the pooling area 112 and this heat may radiate through the vertical sidewall.

When energized, the resistive wire 128 may heat to a temperature in the order of a hundreds of degrees Fahrenheit, the energized resistive wire 128 may then impart thermal energy on to the heating element assembly through convective and the conductive heating. The resulting thermal transfer from the heating element assembly, may result in the heating element assembly at the distal end, to reach a predetermined temperature (500 F to 800 F) for vaporizing of the material for vaporizing disposed proximate to the distal end of the heating element assembly.

In some embodiment the heating element assembly, maybe manufactured from a non-porous ceramic having the resistive heating wire 128 and embedded therein, such as a ceramic heating rod or a stainless steel encased heating rod.

The vertical sidewall 110w may extend from the proximal end 110p towards the distal end 110d, the vertical sidewall 110w other than for supporting the phyto material for vaporizing 419 and for contacting of the phyto material for vaporizing 419 disposed proximate the pooling area 112. The vertical sidewall 110w may be non-porous proximate the proximal end of the heating element assembly to facilitate a flow of material distally along the vertical sidewall 110w towards the distal end

In some embodiments, the vertical sidewall 110w, may be manufactured from a different material at the distal end and a different material at the proximal end of the heating element assembly. The heating may facilitate of thermal energy from the distal end of the heating element assembly to propagate less thermal energy towards the heating element assembly arm when a thermal insulating material is used proximate the distal end of the vertical sidewall 110w. Materials such as ceramic or glass may be envisaged.

In some embodiments, the heating element assembly 110 may be manufacturer from a borosilicate glass or a quartz glass, with the resistance heating element wire 128 heating shielded by the glass or ceramic or metal or other housing 101 such that it the heating element wire is other than in fluid contact with the phyto material for vaporizing 419 and in thermal communication with the material for vaporizing 419, which may be disposed in the pooling area. Having the heating element wire other than contacting the phyto material 419 may facilitate a longer lifetime of the heating element wire as well as to preserve a flavor of vapor 421 emitted from the device. The vertical sidewall 110w may surround the heating element wire. The heating element assembly 110 may comprise silicon carbide or borosilicate glass or maybe manufactured from metal or a non-porous material and in some embodiments it may surround the heating element wire. In some embodiments the heating element wire may be formed from a resistive film sintering process.

The heating element assembly may include a temperature sensor 170 thermally coupled for detecting a temperature of the heating element wire. The temperature sensor 170 may have a temperature signal output port. Typically, the temperature signal is based on a resistance of the temperature sensor 170 and the resistance may vary inversely with respect to the temperature being sensed by the temperature sensor 170. The coupling arm may include coupling arm contacts 186 for electrically coupling of the heating element with a control circuit assembly and for electrically coupling of the temperature sensor 170 with the control circuit assembly. The heating element assembly may be coupled to a first end 117a of the coupling arm and a second end 117b of the coupling arm may be releasably coupled with the housing 101 through an electrical connection port 118 that comprises the coupling arm contacts 186.

In some embodiments the heating element assembly 110b may not directly contact the phyto material for vaporizing 419 when in a resting state. The phyto material for vaporizing 419 may pool within the pooling area and may have a surface that other than contacts heating element assembly 110b. In some embodiments a heating element assembly pooling area facing end 110f disposed proximate the distal end of the heating element assembly may be facing the phyto material for vaporizing 419 and a surface of the heating element assembly pooling area may be approximately parallel with a surface of the phyto material for vaporizing 419 when in the resting state. In some embodiments in a resting state where the phyto material for vaporizing 419 has had its viscosity reduced because of absorption of heat from the heating element assembly, the surface of the phyto material for vaporizing 419 may be in a plane at angle with a heating element assembly pooling area facing end. The heating element assembly pooling area facing end may following a same curvature as the phyto material support surface or it may be flat and approximately horizontal.

In use, in some embodiments, with reference to FIG. 1E, the distal end of the housing 101 may be configured to engage a water pipe through a water pipe engagement port coupled with the support unit 191 so that the vaporizing assembly may be supported on by the water pipe downstem and the distal opening 101d communicates with a lumen of the water pipe downstem where vapor propagates through a waterpipe fluid pathway through to an inhalation aperture. Generating a low pressure at the inhalation aperture may facilitate air 555 to flow through the vaporization assembly 100 and vapor to flow into the inhalation aperture.

When electrical power is controllably applied to the heating element assembly, a controllable airflows stream 556 may be directed towards the phyto material in the pooling area from the airflow directing member 189 may result in the phyto material in the pooling area to splash against the vertical sidewall 110w. The controllable airflows stream 556 may be disturbed by the pooled material and becomes turbulent and may cause turbulent flow may then facilitate condensation of the phyto material extract and the phyto material extract vapor on the heating element assembly vertical sidewall 110w and more specifically on the sidewall proximate the distal end thereof as well as the phyto material support surface 102 and for the condensed material for vaporization to gravitationally flow into the pooling area 112 for subsequent interaction with by the user.

The airflow may enter through the airflow directing member 189 and the user may user their finger or another object to restrict the airflow as well as to potentially pulse the incoming airflow 555 that is entering the at least a vapor aperture 126. The airflow directing member 189 may be formed from a hollow tubular construction that may generally be oriented parallel with the heating element assembly 110.

Referring to FIG. 1A, the lid assembly 120 may be rotated about the heating element assembly longitudinal axis 110z for directional air control 556 for the incoming air moving around the phyto material for vaporizing 419 controllably towards the vertical sidewall 110w, where the lid assembly 120 may be rotated for altering airflow characteristics and interacting with the vapor for being able to controllable alter airflow along the vertical sidewall 110w, the pooling area 112, the heating element assembly gap 111, the heating element assembly and between the heating element assembly and the vertical sidewall 110w. Through rotating of the lid assembly 120 about the heating element assembly longitudinal axis 110z

When observed from a top view, the lid assembly 120 may rotate at least 120 degrees or in some cases 180 degrees for potential directional air control for the controllable airflows stream 556.

The pooled phyto material extract 419 may then contact the heating element assembly and form a film on its outer sidewall and the film may then be heated and release vapor. Excess material that does not result the creation of vapor may then gravitationally flow into the pooling area for subsequent disturbance through incoming airflow or thorough mechanical stimulation by the user.

Having a phyto material extract film contacting the heating element assembly may facilitate a decreased heating time for the heating element assembly when the heating element assembly is other than submerged in the pooled phyto material extract and when it is used for heating of the heating pooled phyto material extract it may be largely heated through convective means and less heated through conductive means.

Having a larger amount of phyto material extract contacting the heating element assembly may facilitate an increased heating time for the heating element assembly when at least a portion of the heating element assembly is submerged in the pooled phyto material extract and when it is used for heating of the heating pooled phyto material extract it may be largely heated through conductive means and less heater through convective means.

In some embodiments, a user may utilize a poking tool proximate the elongated member and for this tool to be inserted into the housing 101 within the cavity from the proximal end and they use this tool to stir the phyto material located in the pooling area. In some embodiments the incoming airflow from an external environment 555 may be directed through the airflow member 189 and to be steered within the housing proximate the pooling area to direct incoming air towards the pooling area.

FIG. 1E illustrates a support unit 119 may include a housing and comprising a user interface to 120, the user interface may facilitate for the user to change a temperature of the heating element assembly or a duration for which the heating element assembly is powered. User interface may also include at least a display assembly (OLED) and a button assembly and components may form part of the control circuit or control assembly. The housing 101 may be elongated and having a housing length 101h1 that is longer than a housing diameter 101hd. The user interface may include buttons and dials for the controllable adjusting of electrical power provided to the heating element assembly.

In some embodiments, a battery or power source may be disposed within the housing 101 or attached with the housing 101 or formed as a separate part and a releasably connectable to the housing 101, an example of which is shown in FIG. 1E.

Referring to FIG. 1B and FIG. 1E a connection port 218 may be provided and comprises at least two releasable electrical contacts for coupling of the heating element assembly 110 to an external control circuit assembly as part of the support unit 119. In some cases the connection may have three or four or five electrical connections that may allow for determining a type of module connected with the support unit 119.

The support unit 119 may include the fluid pathway 115 wherein the vapor 421 being provided to the end user may flow along the fluid pathway 115.

An energy source may be coupled to a control circuit (FIG. 1E) for control be providing of electrical energy to the heating element assembly in accordance with embodiments of the invention and may use thermal heating profiles that may be represented as pulse width modulation profiles.

In some embodiments, incoming airflow from an external environment 555 flowing through the incoming airflow channel 189 may form a vortex about the heating element assembly between the outer sidewall and the phyto material support surface 102 when propagating through the airflow directing member 189 that receives the incoming airflow from an external environment 555 when a suction is generated at the vapor and coupling port at the distal end of the housing.

In some embodiments heating element assembly may be provided with a rotary power coupling for rotationally coupling of the heating element assembly with the support arm 117, the rotary power coupling may facilitate a rotation of the heating element assembly about its longitudinal axis and radially extending protrusions may be formed on outside surface of the heating element assembly facing the phyto material support surface and through rotating of the heating element assembly, the radially extending protrusions may facilitate a mechanical interaction with the phyto material disposed in the pooling area.

This may be a further method by which the user heating able to interact with the phyto material extract disposed on the phyto material support surface. Wipers may facilitate lifting of the material from the distal ends towards the proximal end of the housing 101 and may furthermore operate in tandem with directed airflow into the pooling area. These processes may facilitate increasing of vapor production. The protrusions or wipers or vanes may be connected with the heating element assembly.

In some embodiments, the heating element may include a wound wire coil that may have a low thermal inertia as able to attain approximately 700 degrees Fahrenheit within the predetermined temperature and approximately 5 to 10 seconds.

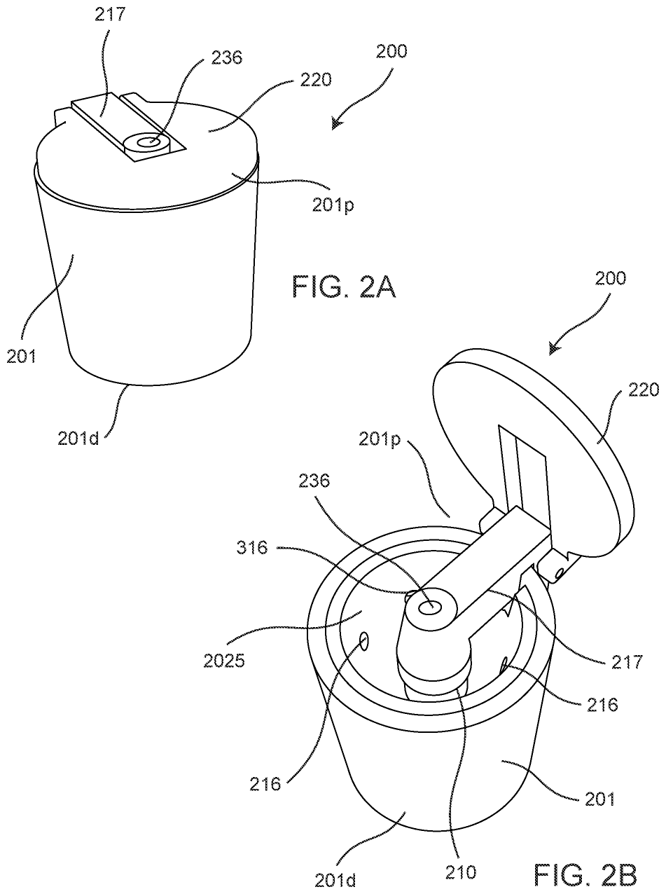

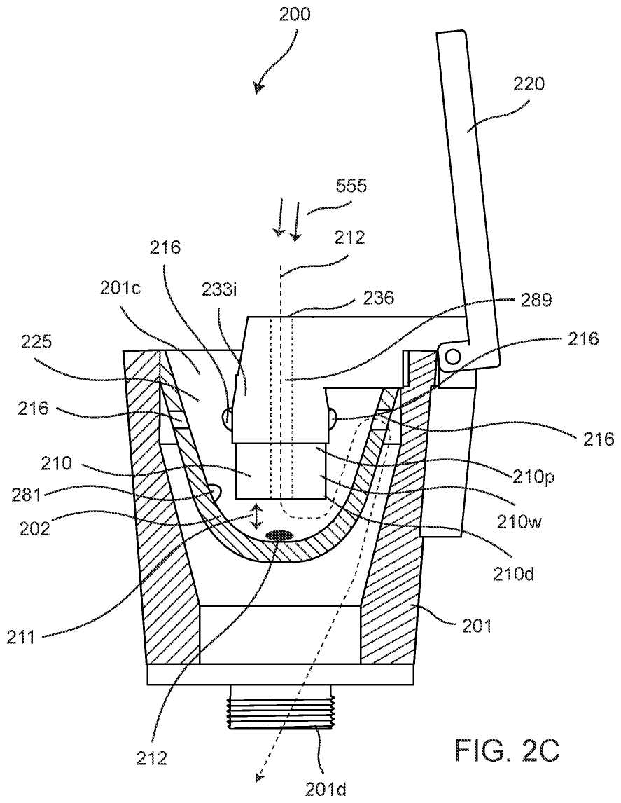

Generally, FIGS. 2A through 2C illustrate a vaporizing assembly 200 in accordance with a second embodiment of the invention. The vaporizing assembly 200 in accordance with the second embodiment of the invention may be formed from a housing 201 having a proximal end 201a and a distal end 201d, the distal end 201d may be disposed lower than the proximal end 201p and the proximal end 201p comprising a proximal opening 201c and at the distal end comprising a distal opening 201d. Other frictional engagements mechanisms are also envisaged for frictionally engaging of the lid assembly 220 with the proximal opening 201c.

FIG. 2A illustrates the lid assembly 220 which may be coupled with a hinge with the housing 201 and may be shown in a closed position. FIG. 2B. illustrates the lid assembly 220 which may be coupled with the hinge to the housing 201 and may be shown in a closed position.

Whether the lid assembly is in the opened or closed position the coupling arm 217 remains coupled with the housing 201 and the heating element assembly remains in its position relation to the material support surface 202. From a safety perspective it is advantageous to have the heating element assembly contained within the housing so that potential burns are avoided for the user through touching of the heating element assembly.

In some embodiments, a vapor aperture 216 (FIG. 2B, 2C) may be formed approximately in the sidewall 202s proximate the proximal end of the material support surface 202. Having the vapour apertures, form proximal the proximal end of the phyto material support surface 102 facilitates for material for vaporizing to be loaded into the housing 201 and it may be contained by heating the material support surface 102. Having the vapour apertures disposed proximally from the distal end of the phyto material support surface 202 facilitates a larger and deeper pooling area for the phyto material extract 419.

Referring to FIG. 2B, a the phyto material support surface 202 may be provided facing the proximal end 201a of the housing 201 and accessible through the proximal opening 201c and fluidly coupled therewith, the phyto material support surface 202 may be configured to support a phyto material for vaporizing 419 and disposed distally below the proximal opening 201c and in fluid communication with the proximal opening 201c for facilitating loading of material for vaporization through the proximal opening 201c and for contacting of the phyto material support surface 202.

FIG. 2B illustrates the phyto material support surface 202 may be formed from at least a partial paraboloid surface that comprises a quadric surface that has one axis of symmetry wherein a proximal end of the phyto material support surface 202 may have an outer diameter greater than an outer diameter of a distal end of the phyto material support surface 202 and the phyto material support surface 202 may taper from the proximal to the distal end comprising the pooling area 212 that may be disposed at a gravitational low point of the phyto material support surface 202 or at a vertex of the paraboloid surface.

A heating element assembly 210 may be disposed between the proximal opening 201c and the phyto material support surface 202 the heating element assembly 210 comprising a proximal end 210a and a distal end 210b, the distal end 210b of the heating element assembly 210 being proximally spaced from the phyto material support surface 202 through a heating element assembly gap 211. The heating element assembly gap 211 may be maintained by the coupling arm 217.

The phyto material support surface 202 may be for receiving of a phyto material for vaporizing 419 and the material may be gravitationally induced to flow towards a pooling area 212, the phyto material for vaporizing 419 proximate the pooling area 212 for at least one of being proximate the distal end of the heating element assembly 210 and for contacting the heating element assembly 210 and for having at least a portion of the phyto material for vaporizing 419 being heated to a predetermined temperature to result in a vapor 421 to be emitted from the material for vaporizing 419. The pooling area 212 may be approximately centrally disposed with respect to the phyto material support surface 202.

In some embodiments a thermal insulating member 233i may be disposed between various materials forming an elongated wall 210w of the heating element assembly. In some embodiments the heating element assembly 210 may have a coaxial airflow channel 289 for receiving of ambient air 555 from an outside environment for flowing towards the pooling area 212, when in use. The airflow channel 289 may propagate from the proximal end 210p through an ambient air input port 236 to the distal ends 210d of the heating element assembly 212 and may controllably direct airflow towards the pooling area 212 and may allow for a controllable ambient air 555 restriction that may be user controllable for controlling a flow rate of ambient air 555 entering interacting with the phyto material for vaporizing 419 proximate the pooling area 212. A fluid pathway 215 may be formed within the housing 201 from the pooling area 212 through at least a vapor aperture 216 and towards the distal opening 201d at the distal end of the housing 201, wherein the vapor 421 flows along the fluid pathway 215. In some embodiments the fluid pathway 215 is at an angle with the heating element assembly 210.

In some embodiments, the heating element assembly 210 may be manufacturer from a porous ceramic material and having a resistance heating element wire embedded therein such that it the heating element wire is in fluid contact with the phyto material for vaporizing 419 when the with the phyto material for vaporizing 419 is absorbed by the porous ceramic material. The vertical sidewall 210w may surround the heating element wire. The heating element assembly 210 may comprise an annular heating element, such as a heating coil, with the coaxial airflow channel 289 propagating axially therethrough.

In some embodiments, the annular heating element may include a non-porous ceramic annular heating element manufactured using a sintering process whereby the with the coaxial airflow channel 289 may propagate through a center of the non-porous ceramic annular heating element. The heating element assembly may reach temperatures of approximately 500 to 800 degrees Fahrenheit. Having the heating element assembly disposed within the housing 201 when the lid heating removed, may provide for the user not to get burnt on the heating element assembly post when inserting of material for vaporizing into the housing 201.

In some embodiments material pooling area may make contact with the heating element assembly distal end and may under the influence of directed airflow may result in splashing of material on to the outer surface or sidewall of the heating element assembly. In some embodiments, ribs proximate or protrusions 281 disposed on a side facing of the phyto material support surface 202 may facilitate material contact with the heating element assembly as air is flowing about the heating element assembly and the phyto material support surface.

In some embodiments the heating element assembly may be movable in an proximal distal direction to vary the gap between the distal end of the heating element assembly and the proximally facing phyto material support surface 202.

The gap may be formed between the phyto material support surface 202 and on the other side has a distal end of the heating element assembly. When the lid is opened and the phyto material support surface 202 is exposed and allows for material to be inserted into the housing 201 between the phyto material support surface 202 and the heating element assembly, the material then slides under gravity along the phyto material support surface 202 and towards the pooling area. The material found within the pooling area does not have any direct heat source that heats the applied at the bottom of the MSS. Resting material that is in the pooling area may cool as such it may not be directly heated be heated by the heating element assembly to temperature sufficient to cause vaporizing unless the material is in direct thermal contact with the heating element assembly and that thermal contact results in the material to receive sufficient thermal energy to cause vapor to be emitted therefrom.

In some embodiments interacting with the material in the pooling area this may result in the material to physically contact and outside of the heating element assembly. The heating element sidewall, may have a temperature sufficient to cause vaporizing of the material and vapors are emitted from the surface and travel to the vapor apertures.

In some embodiments upon disabling an operation of the heating element assembly, material that may be found on an outside surface of the heating element assembly may under the force of gravity propagate from the proximal end towards the distal end of the heating element assembly and may drip into the pooling area.

In some embodiments, the process of directing air flow towards the pooling area may cause the pooled material to percolates onto and about the heating element assembly and they also for the condensation on the phyto material support surface or the heating element assembly.

In some embodiments, the heating element assembly may radiate heat as well as provide for light such that and then user may be able to see, or visually determine, a temperature of the heating element assembly through a color temperature of the heating wire.

Through suspending of the heating element assembly above the phyto material support surface, phyto material extract disposed on the phyto material support surface is subjected to heat from an upper side or proximal side and other than subjected to heat from a distal side. the heating element assembly. Heating from the top as opposed to the bottom may result in the material placed on the non heated support surface to potentially burn less as opposed to potentially burning when bottom heated. It is therefore advantageous to provide for a system and method of vaporizing of phyto material extracts that does not heat the phyto material extracts from the bottom that causes materials to be burned or overheated.

A further advantage of the embodiments of the invention is provided by controlling incoming air contacting the phyto material in the pooling area and for the incoming air to mechanically interact with the pooled phyto material extract to mechanically disturb this extract for creating droplets or aerosol of the phyto material extract that is proximate the heating element assembly.

While the above description describes features of example embodiments, it will be appreciated that some features and/or functions of the described embodiments are susceptible to modification without departing from the spirit and principles of operation of the described embodiments. For example, the various characteristics which are described by means of the represented embodiments or examples may be selectively combined with each other. Accordingly, what has been described above is intended to be illustrative of the claimed concept and non-limiting. It will be understood by persons skilled in the art that other variants and modifications may be made without departing from the scope of the invention as defined in the claims appended hereto. The scope of the claims should not be limited by the preferred embodiments and examples, but should be given the broadest interpretation consistent with the description as a whole.

What I claim is:

1. A suspended thermal radiator vaporizing assembly comprising:
a housing having a proximal end and a distal end, the distal end disposed lower than the proximal end and the proximal end comprising a proximal opening and at the distal end comprising a distal opening;
a phyto material support surface facing the proximal end of the housing and accessible through the proximal opening and fluidly coupled therewith, the phyto material support surface configured to support a phyto material extract and disposed distally below the proximal opening and in fluid communication therewith,
a heating element assembly disposed between the proximal opening and the phyto material support surface the heating element assembly comprising a proximal end and a distal end, the distal end of the heating element assembly being proximally spaced from the phyto material support surface through a heating element assembly gap, the distal end of the heating element assembly for providing of at least one of convective and conductive heating to the phyto material extract;
the phyto material support surface for receiving of the phyto material extract for vaporizing for having at least a portion of the phyto material extract gravitationally induced to flow towards a pooling area, the phyto material extract proximate the pooling area for thermally contacting the heating element assembly and for being heated to a predetermined temperature to result in a vapor to be emitted from the phyto material extract;
a fluid pathway formed within the housing and fluidly coupled with the pooling area through at least a vapor aperture and towards the distal opening at the distal end of the housing, wherein, in use, the vapor flows along the fluid pathway towards the distal end of the housing.

2. A vaporizing assembly according to claim 1 wherein the phyto material support surface comprises at least a partial paraboloid surface that comprises a quadric surface that has one axis of symmetry wherein a proximal end of the phyto material support surface has an outer diameter greater than an outer diameter of a distal end of the phyto material support surface and the phyto material support surface tapers from the proximal to the distal end comprising the pooling area.

3. A vaporizing assembly according to claim 1 wherein the heating element assembly is coupled to the housing with a coupling arm that supports the heating element assembly for providing the heating element assembly gap with the heating element distal end proximate the pooling area.

4. A vaporizing assembly according to claim 3 wherein the heating element assembly gap may be about 0.5 mm.

5. A vaporizing assembly according to claim 1 comprising a lid assembly, the lid assembly for being releasably coupled with the housing, wherein the heating element heating disposed between the lid assembly and the phyto material support surface when the lid is coupled with the housing the proximal opening may be covered for other than inserting of phyto material extract into the vaporizing assembly.

6. A vaporizing assembly according to claim 1 comprising a lid assembly, the lid assembly for being hinged the housing, wherein the lid assembly for operating between a covered position where the lid assembly provides for reduced external airflow from an outside environment into the at least a vapor aperture and an uncovered position where the external airflow may flow from an outside environment into the at least a vapor aperture.

7. A vaporizing assembly according to claim 1 comprising:
a lid assembly, the lid assembly for being releasably coupled with the housing for being in an uncoupled state with the housing for loading of phyto material extract into a phyto material receiving cavity;
the lid assembly for being releasably coupled with the housing for being in a coupled state with the housing for having the lid assembly for rotating about a heating element assembly longitudinal axis;
and the lid assembly comprising an airflow directing member for receiving of external air from an outside environment into at least a vapor aperture and a vapor output aperture disposed within the phyto material receiving cavity and proximate the phyto material support surface whereby air deflects from the vapor output aperture upon impacting of the phyto material support surface and a controllable airflows stream may be directed towards the phyto material in the pooling area.

8. A vaporizing assembly according to claim 7 wherein the heating element assembly comprises a vertical sidewall extending from the proximal end towards the distal end, the vertical sidewall comprising a non porous surface for other than for supporting the phyto material extract and comprising a resistive heating wire embedded therein.

9. A vaporizing assembly according to claim 7 wherein the heating element assembly comprises a vertical sidewall extending from the proximal end towards the distal end, the vertical sidewall comprising a porous surface for and comprising a resistive heating wire embedded therein.

10. A vaporizing assembly according to claim 1 comprising a support unit for releasably coupling with the distal end of the housing, wherein the support unit is for providing of electrical power to the heating element assembly, the support unit for engaging a water pipe downstem so that the vaporizing assembly heating supported on by the water pipe downstem and the distal opening communicates with a lumen of the water pipe downstem.

11. A vaporizing assembly according to claim 1 wherein the heating element assembly comprises a vertical sidewall extending from the proximal end towards the distal end, the vertical sidewall other than supporting the material for vaporizing thereon, wherein the vertical sidewall is manufacture from a non porous material, wherein a resistive heating wire is contained within vertical sidewall that surrounds the resistive heating wire.

12. A vaporizing assembly according to claim 3 comprising a lid assembly, the lid assembly for being releasably coupled with the housing, wherein when the lid assembly is other than coupled with the housing, the coupling arm remains coupled with the housing and the heating element assembly remains in a fixed position in relation to the phyto material support surface.

13. A vaporizing assembly according to claim 3 wherein the heating element assembly is coupled to a first end of the coupling arm and a second end of the coupling arm heating releasably coupled with the housing through an electrical connection port.

14. A vaporizing assembly according to claim 13 wherein the a connection port comprises at least two releasable electrical contacts for coupling of the heating element assembly to an external control circuit assembly.

15. A suspended thermal radiator vaporizing assembly comprising:
disposing and supporting of the vaporizer assembly into a downstem of a water pipe so that a portion of the housing of the vaporizer assembly heating supported by a water pipe downstem;
inserting phyto material extract through a proximal opening with the phyto material extract touching the phyto material support surface and at least a portion of the inserted phyto material extract for gravitationally flowing along the phyto material support surface towards a pooling area;
providing a heating element assembly spaced proximally from the phyto material support surface through a heating element assembly gap,
applying of electrical energy to the heating element assembly, the heating element assembly for providing of thermal energy to material for vaporizing proximate the heating element assembly through at least one of convective and conductive heating so that at least a portion of the material for vaporizing heating vaporized into a vapor; and
drawing the vapor along a vapor path proximate the phyto material support surface and a distal end of the heating element assembly and through the housing and into the downstem of the water pipe, wherein in use the phyto material support surface other than achieves a temperature sufficient to result in vaporization of the phyto material extract.

16. A suspended thermal radiator vaporizing assembly according to claim 15 comprising:
providing a lid assembly rotationally coupled in relation with the phyto material support surface for rotating about a heating element assembly longitudinal axis;
the lid assembly comprising an airflow directing member for receiving of external air from an outside environment into at least a vapor aperture and a vapor output aperture disposed within a phyto material receiving cavity and proximate the phyto material support surface whereby air deflects from the vapor output aperture upon impacting of the phyto material support surface and a controllable airflows stream may be directed towards the phyto material in the pooling area.

17. A suspended thermal radiator vaporizing assembly comprising:
a housing having a proximal end and a distal end, the distal end disposed lower than the proximal end and the proximal end comprising a proximal opening and at the distal end comprising a distal opening;
a phyto material support surface facing the proximal end of the housing and accessible through the proximal opening and fluidly coupled therewith, the phyto material support surface configured to support a phyto material extract for vaporizing and disposed distally below the proximal opening and in fluid communication therewith,
a heating element assembly disposed between the proximal opening and the phyto material support surface the heating element assembly comprising a proximal end and a distal end, the distal end of the heating element assembly being proximally spaced from the phyto material support surface through a heating element assembly gap, the distal end of the heating element assembly for providing of at least one of convective and conductive heating to the phyto material extract;
the phyto material support surface for receiving of the phyto material extract for vaporizing for having at least a portion of the phyto material extract gravitationally induced to flow towards a pooling area, the phyto material extract proximate the pooling area for thermally contacting the heating element assembly and for being heated to a predetermined temperature to result in a vapor to be emitted from the phyto material extract;
a lid assembly, the lid assembly for being releasably coupled with the housing for being in an uncoupled state with the housing for loading of phyto material extract into a phyto material receiving cavity;
the lid assembly for being releasably coupled with the housing for being in a coupled state with the housing for having the lid assembly for rotating about a heating element assembly longitudinal axis;
and the lid assembly comprising an airflow directing member for receiving of external air from an outside environment into at least a vapor aperture and a vapor output aperture disposed within the phyto material receiving cavity and proximate the phyto material support surface whereby air deflects from the vapor output aperture upon impacting of the phyto material support surface and a controllable airflows stream may be directed towards the phyto material in the pooling area; and
a fluid pathway formed within the housing and fluidly coupled with the pooling area through the at least a vapor aperture and towards the distal opening at the distal end of the housing, wherein, in use, the vapor flows along the fluid pathway towards the distal end of the housing.
